(19) United States
(12) Patent Application Publication
(10) Pub. No.: US 2020/0101256 A1
(43) Pub. Date: Apr. 2, 2020

(54) Circular Bladder Oxygen Therapy Exercise System

(71) Applicant: MaxxO2, LLC, Austin, TX (US)

(72) Inventor: Anthony Lee Parasco, Bradenton, FL (US)

(73) Assignee: MaxxO2, LLC, Austin, TX (US)

(21) Appl. No.: 16/590,864

(22) Filed: Oct. 2, 2019

Related U.S. Application Data
(60) Provisional application No. 62/740,287, filed on Oct. 2, 2018.

(51) Int. Cl. A61M 16/12; A61M 16/00

(52) U.S. Cl. CPC A61M 16/12; A61M 2202/0208; A61M 16/0078

(57) ABSTRACT

The invention relates generally to a circular bladder system for exercise with oxygen therapy (EWOT). The circular bladder design supplies, controls, and/or select between two or more airflows with one bladder that is cylindrical and collapsible within a frame; and the other bladder that is mounted atop the frame. The bladder contains different oxygen levels and pressures to simulate different altitudes.

### FIELD OF THE INVENTION

The present invention relates generally to a bladder system for exercise with oxygen therapy (EWOT). More specifically, the present invention is a dual bladder system for supplying, controlling, and/or selecting between at least two airflows with one bladder that is cylindrical, collapsible, and within a frame; and another bladder that is mounted atop the frame.

### BACKGROUND OF THE INVENTION

In present society, EWOT is commonly used as a versatile medical therapy for a wide variety of medical ailments. Currently, most EWOT devices are crude and cumbersome devices that take up too much floor space. Although some EWOT devices could be made with a smaller footprint, these devices are not easily transportable and/or storable. Once these EWOT devices are in place, it can be difficult to transport said devices without completely dismantling said devices. Additionally, some EWOT devices can have difficulty in maintaining positive pressure upon the air bladders utilized to store the oxygen or other similar gases. Also, some EWOT devices only supply a single air source; not allowing in multiple air sources to be used within the same EWOT device.

An objective of the present invention is to provide users with a device that is a dual gas bladder system for EWOT. The present invention intends to provide users with an EWOT device that can supply at least two gasses. The present invention intends to provide users with a selector-switch to manually switch between the desired gas supplies. The present invention intends to provide users with a gas bladder than can maintain positive pressure. The present invention intends to provide users with a device that is reduces the floor space required by other EWOT devices.

### SUMMARY OF THE INVENTION

The present invention is a dual gas bladder system for EWOT. The present invention contains a frame. The present invention contains a primary gas bladder that is compressible when deflated. The walls of the primary gas bladder are attached to the medial surface of the side faces of the frame by a plurality of sliders, which allow the free end (or top end) of the primary gas bladder to rise when primary gas bladder is inflating. The present invention contains a plurality of weights attached to the primary gas bladder. The present invention contains a secondary gas bladder mounted on top of the frame. The present invention contains a primary gas supply and a secondary gas supply connected to the primary gas bladder and the secondary gas bladder at one end of said bladders, respectively. At the other end of the bladders, the present invention contains a three-way valve, which is connected to a breathing mask at the other end.

### BRIEF DESCRIPTION OF THE DRAWINGS

FIG. 1. is a drawing of the present invention.

FIG. 2 is a photo of the frame and first bladder of the present invention.

### DETAIL DESCRIPTIONS OF THE INVENTION

All illustrations of the drawings are to be describing selected versions of the present invention and are not intended to limit the scope of the present invention. All references of user or users pertain to either individual or individuals who would utilize the present invention. All references of EWOT can pertain to exercise with oxygen therapy, exercise with oxygen training, or other similar objects.

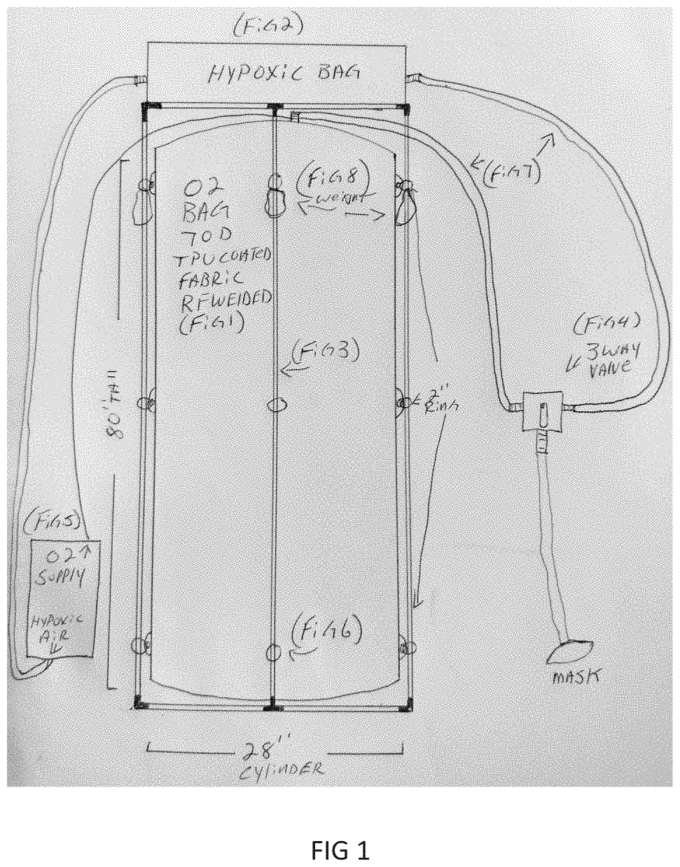

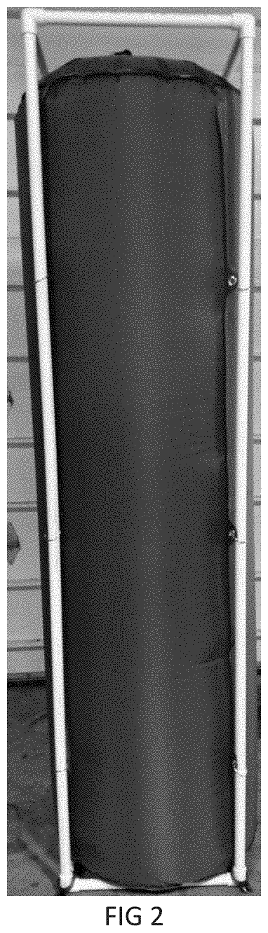

As seen in FIG. 1 through FIG. 2, the present invention is a dual gas bladder system for EWOT. The present invention can be of any shape, size, materials, features, type or kind, orientation, location, quantity, components, and arrangements of components that would allow the present invention to fulfill the objectives and intents of the present invention. The present invention primarily includes a frame, a first bladder, a second bladder, a first gas supply, a second gas supply, a valve, and a breathing mask.

As seen in FIG. 1 through FIG. 2, the present invention contains a frame. The frame can be of any shape, size, materials, features, type or kind, orientation, location, quantity, components, and arrangements of components that would allow the present invention to fulfill the objectives and intents of the present invention. However, it can be preferred that the frame be of a material that is strong, durable, load-bearing, friction-less, tough, and/or lightweight. It can be preferred that the frame be of a shape similar to a hollow, quadrilateral-like shaped object; for instance, as illustrated in FIG. 2. It can be preferred that the frame be of a height that is greater than the length and/or width of the frame. It can be preferred that the frame be of a feature such that the frame can be of a plurality of sectioned members that can be modular and be connected together, as illustrated in FIG. 2, or that the frame be a feature such that the frame can be of a single, rigid, whole structure that can be non-sectioned and/or non-dismantleable. The frame can include a plurality of side members, a plurality of base members, and a plurality of cover members.

As seen in FIG. 1 and in FIG. 2, the frame contains a plurality of side members. The plurality of side members can be of any shape, size, materials, features, type or kind, orientation, location, quantity, components, and arrangements of components that would allow the present invention to fulfill the objectives and intents of the present invention. However, it can be preferred that the plurality of side members be of material and height similar to the material and height of the frame. It is preferred that the quantity of the plurality of side members be such that the quantity of the plurality of side members corresponds with the shape of the frame; for instance, for a frame of a quadrilateral-like shape, the plurality of side members would be of a quantity of at least four. The plurality of side members contains a plurality of sliders. The plurality of sliders can be of any shape, size, materials, features, type or kind, orientation, location, quantity, components, and arrangements of components that would allow the present invention to fulfill the objectives and intents of the present invention. However, it can be preferred that the plurality of sliders be located along the exterior surfaces of the plurality of side members. It can be preferred that the plurality of sliders be of a type or kind, shape, and/or feature similar to rings. It is preferred that the plurality of sliders be attached to the sides of the first bladder by a plurality of grommets or other similar means of attachment.

As seen in FIG. 1 through FIG. 2, the frame contains a plurality of base members and a plurality of cover members. The plurality of base members can be of any shape, size, materials, features, type or kind, orientation, location., quantity, components, and arrangements of components that would allow the present invention to fulfill the objectives and intents of the present invention. However, it can be preferred that the plurality of base members be similar to the plurality of side members in material and quantity. It is preferred that the plurality of base members be attached to the bottom ends of the plurality of base members, as illustrated in FIG. 1 through FIG. 2. The plurality of cover members can be of any shape, size, materials, features, type or kind, orientation, location, quantity, components, and arrangements of components that would allow the present invention to fulfill the objectives and intents of the present invention. However, it can be preferred that the plurality of cover members be identical to the plurality of base members in shape, size, materials, features, type or kind, quantity, components, and arrangements of components. It is preferred that the plurality of cover members be attached to the top ends of the plurality of base members, as illustrated in FIG. 1 through FIG. 2.

As seen in FIG. 1 through FIG. 2, the present invention contains a first bladder. The first bladder can be of any shape, size, materials, features, type or kind, orientation, location, quantity, components, and arrangements of components that would allow the present invention to fulfill the objectives and intents of the present invention. However, it can be preferred that first bladder be of a material that is durable, strong, tear-resistant, tough, compressible, lightweight, and/or medical-grade. It can be preferred that the first bladder be of a shape similar to a hollow, cylindrical-like shaped object. It is preferred that the first bladder be of an overall size similar to the size of the inside/chamber of the frame, as illustrated in FIG. 1 through FIG. 2. It can be preferred that the first bladder be of a height similar to the height of the frame. It can be preferred that the bottom of the first bladder be mounted to the bottom of the frame such that the top of the first bladder can raised when the first bladder is inflated and/or lowered when the first bladder is deflated. It is preferred that the sides of the first bladder be attached to the plurality of sliders by a plurality of grommets or other similar means of attachments. The first bladder primarily includes a first inlet, a first outlet, and a plurality of weights. The first inlet can be of any shape, size, materials, features, type or kind, orientation, location, quantity, components, and arrangements of components that would allow the present invention to fulfill the objectives and intents of the present invention. However, it can be preferred that first outlet be connected to the first gas supply. The first outlet can be of any shape, size, materials, features, type or kind, orientation, location, quantity, components, and arrangements of components that would allow the present invention to fulfill the objectives and intents of the present invention. However, it can be preferred that first outlet be connected to the valve.

As seen in FIG. 1, the first bladder contains a plurality of weights. The plurality of weights can be of any shape, size, materials, features, type or kind, orientation, location., quantity, components, and arrangements of components that would allow the present invention to fulfill the objectives and intents of the present invention. However, it can be preferred that plurality of weights be located at top end of the sides of the first bladder. It can be preferred that the plurality of weights be attached to the topmost layer of the plurality of sliders, as illustrated in FIG. 1.

As seen in FIG. 1, the present invention contains a second bladder. The second bladder can be of any shape, size, materials, features, type or kind, orientation, location, quantity, components, and arrangements of components that would allow the present invention to fulfill the objectives and intents of the present invention. However, it can be preferred that second bladder be of a material similar to the material of the first bladder. It can be preferred that the second bladder be of a shape similar to a hollow, quadrilateral-like shaped object. It is preferred that the second bladder be of a floor space size similar to the size of the top face of the frame. It can be preferred that the bottom face of the second bladder be mounted to the top face of the frame. The second bladder primarily includes a second outlet and a second inlet. The second inlet can be of any shape, size, materials, features, type or kind, orientation, location, quantity, components, and arrangements of components that would allow the present invention to fulfill the objectives and intents of the present invention. However, it can be preferred that second inlet be connected to the second gas supply. The second outlet can be of any shape, size, materials, features, type or kind, orientation, location, quantity, components, and arrangements of components that would allow the present invention to fulfill the objectives and intents of the present invention. However, it can be preferred that second outlet be connected to the valve.

As seen in FIG. 1, the present invention contains a first gas supply and a second gas supply. The first gas supply can be of any shape, size, materials, features, type or kind, orientation, location, quantity, components, and arrangements of components that would allow the present invention to fulfill the objectives and intents of the present invention. However, it is preferred that first gas supply be connected to the first inlet of the first bladder. It can be preferred that the first gas supply contain a supply of oxygen gas or a similar object. The second gas supply can be of any shape, size, materials, features, type or kind, orientation, location, quantity, components, and arrangements of components that would allow the present invention to fulfill the objectives and intents of the present invention. However, it is preferred that the second gas supply be connected to the second inlet of the second bladder. It can be preferred that the second gas supply contain a supply of a hypoxic air or a similar object.

As seen in FIG. 1, the present invention contains a valve. The valve can be of any shape, size, materials, features, type or kind, orientation, location, quantity, components, and arrangements of components that would allow the present invention to fulfill the objectives and intents of the present invention. However, it can be preferred that valve be of a material compatible with the material of the first bladder and/or second bladder. It is preferred that the valve be of a type or kind similar to a manual three-way valve. It is preferred that one intake of the valve be connected to the first outlet and the other intake of the valve be connected to the second outlet, while the outtake of the valve be connected to the mask. Alternative embodiments of the present invention could allow for the valve to be of an automatic three-way valve with a remote-control access or similar.

As seen in FIG. 1, the present invention contains a mask. The mask can be of any shape, size, materials, features, type or kind, orientation, location, quantity, components, and arrangements of components that would allow the present invention to fulfill the objectives and intents of the present invention. However, it can be preferred that mask be of a material similar to and/or compatible with the material of the first bladder and/or second bladder. It can be preferred that the mask be of a type or kind similar to a breathing mask or other similar objects. It is preferred that the mask be attached to the outtake of the valve, as illustrated in FIG. 1.

Although the invention has been explained in relation to its preferred embodiment, it is to be understood that many other possible modifications and variations can be made without departing from the spirit and scope of the invention.

I claim:

1. A dual bladder system for supplying gases while exercising, consisting of:
a circular collapsible bladder containing a first gas;
a second bladder containing a second gas;
a mixer coupled to the collapsible bladder and the second bladder, wherein the mixer has a selector switch for supplying the gases to a user.

2. The dual bladder system of claim 1, wherein the first gas and the second gas contain oxygen.
